(19) United States
(12) Patent Application Publication
(10) Pub. No.: US 2008/0075786 A1
(43) Pub. Date: Mar. 27, 2008

(54) Antimicrobial agents derived from cream

(75) Inventors: Michel Lange, Quebec (CA); Martin Clement, St-Ambroise-de-Kildare (CA); Jessy Tremblay, Montreal (CA); Jacques Thibodeau, Outremont (CA); Pierre Belhumeur, Laval (CA); Julie Shareck, Dollard-Des-Ormeaux (CA)

Correspondence Address: GORDON & REES LLP, 101 WEST BROADWAY, SUITE 1600, SAN DIEGO, CA, 92101, US

(73) Assignee: Universite de Montreal, Montreal (CA)

(21) Appl. No.: 11/580,633

(22) Filed: Oct. 13, 2006

(30) Foreign Application Priority Data
Sep. 26, 2006 (CA) 2,561,104

(51) Int. Cl. A61K 35/20; A61K 31/202

(52) U.S. Cl. 424/535; 514/560

(57) ABSTRACT

Methods for interfering with the growth of fungi by exposing the fungi to an antimicrobial agent derived from cream, and more particularly, to methods for treating human fungal pathogens such as Candida albicans and Aspergillus fumigatus, through exposing the fungi to free fatty acids with antimicrobial activity derived from cream.

### FOREIGN PRIORITY

The present application claims foreign priority under 35 U.S.C. 119(b) and 37 C.F.R. 1.55(a) to Canadian Patent Application entitled ANTIMICROBIAL AGENTS DERIVED FROM CREAM, filed Sep. 26, 2006, Serial Number ______.

### FIELD OF THE INVENTION

The present invention relates to methods and pharmaceutical compositions for interfering with the growth of fungi by exposing the fungi to an antimicrobial agent derived from cream, and more particularly, to methods for treating human fungal pathogens such as Candida albicans and Aspergillus fumigatus, through exposing the fungi to free fatty acids with antimicrobial activity derived from cream.

### BACKGROUND OF THE INVENTION

Invasive fungal infections (“IFI”) have emerged as a considerable public health problem in recent decades, causing high morbidity and mortality rates among AIDS patients and other immunocompromised individuals ((Clark, T. A. and Hajjeh, R. A. 2002. Recent trends in the epidemiology of invasive mycoses. Curr. Opin. Infect. Dis. 15: 569-574.); Kullberg, B. J. and Oude Lashof, A. M. 2002. Epidemiology of opportunistic invasive mycoses. Eur. J. Med. Res. 7: 183-191.)). Recent epidemiological surveys have indicated that the most common clinical presentation of IFI is due to Candida species, with Candida albicans (“C. albicans”) accounting for the majority of cases (Jasser and Elkhizzi, 2004; Hajjeh, R. A., Sofair, A. N., Harrison, L. H., Lyon, G. M., Arthington-Skaggs, B. A., Mirza, S. A., Phelan, M., Morgan, J., Lee-Yang, W., Ciblak, M. A., Benjamin, L. E., Sanza, L. T., Huie, S., Yeo, S. F., Brandt, M. E. and Warnock, D. W. 2004. Incidence of bloodstream infections due to Candida species and in vitro susceptibilities of isolates collected from 1998 to 2000 in a population-based active surveillance program. J. Clin. Microbiol. 42: 1519-1527.; Tortorano, A. M., Caspani, L., Rigoni, A. L., Biraghi, E., Sicignano, A., and Viviani, M. A. 2004. Candidosis in the intensive care unit: a 20-year survey. J. Hosp. Infect. 57: 8-13). However, non-albicans Candida species, as well as rare fungal etiologic agents such as Fusarium spp., Peacilomyces lilacinus and Pseudallescheria boydii, are also being implicated ((Cimon, B., Carrere, J., Vinatier, J. F., Chazalette, J. P., Chabasse, D., and Bouchara, J. P. 2000. Clinical significance of Scedosporium apiospermum in patients with cystic fibrosis. Eur. J. Clin. Microbiol. Infect. Dis. 19: 53-56.); Groll, A. H. and Walsh, T. J. 2001. Uncommon opportunistice fungi: new nosocomial threats. Clin. Microbiol. Infect. 7 Suppl 2: 8-24.; Idigoras, P., Perez-Trallero, E., Pineiro, L., Larruskain, J., Lopez-Lopategui, M. C., Rodriguez, N., and Gonzalez, J. M. 2001. Disseminated infection and colonization by Scedosporium prolificans: a review of 18 cases, 1990-1999. Clin. Infect. Dis. 32: E158-E165; Redding, S. W. 2001. The role of yeasts other than Candida albicans in oropharyngeal candidiasis. Curr. Opin. Infect. Dis. 14: 673-677.)). Additionally, invasive aspergillosis, most commonly due to Aspergillus fumigatus (“A. fumigatus”), has reached alarming proportions in recent decades, accounting in recent years for the majority of reported IFI in some institutions ((Denning, D. W. 1998. Invasive aspergillosis. Clin. Infect. Dis. 26: 781-803.); Latge, J. P. 1999. Aspergillus fumigates and aspergillosis. Clin. Microbiol. Rev. 12: 310-350)). This epidemiological data indicates that the spectrum of pathogenic fungi causing IFI in humans is changing over time.

Despite the extensive utilization of antifungal therapies, the treatment of IFI remains elusive as some antifungal agents cause toxicity and exhibit a poor absorption in addition to interacting with other drugs (Patterson, T. F. 2001. Invasive mycoses: management and unmet medical needs. Curr. Opin. Infect. Dis. 14: 669-671; Kontoyiannis, D. P. and Lewis, R. E. 2002. Antifungal drug resistance of pathogenic fungi. Lancet 359: 1135-1144). Additionally, an increasing number of antifungal agents are becoming less effective since some pathogenic fungi are developing resistance mechanisms (Kontoyiannis and Lewis, 2002; Sanglard, D. and Odds, F. C. 2002. Resistance of Candida species to antifungal agents: molecular mechanisms and clinical consequences. Lancet Infect. Dis. 2: 73-85). While recent studies introduced newer promising antifungal agents and alternative immuno/combinations therapies, there is a need for new and non-toxic antifungal agents with a wide spectrum of action, especially as emerging pathogenic fungi seem to be particularly resistant to current antifungal agents (Kontoyiannis and Lewis, 2002).

Bovine whey traditionally defined as a by-product of the cheese-making industry is now recognized as a functional aliment with several interesting properties including antioxidative, anticarcinogenic and antimicrobial activity (Walzem, R. L., Dillard, C. J., and German, J. B. 2002. Whey components: millennia of evolution create functionalities for mammalian nutrition: what we know and what we may be overlooking. Crit Rev. Food Sci. Nutr. 42: 353-375). While a number of studies have indicated that several proteins present in bovine whey exhibit antimicrobial activity against bacteria, virus and fungi, other factors could be involved (Shah, N. P. 2000. Effects of milk-derived bioactives: an overview. Br. J. Nutr. 84 Suppl 1: S3-10; Isaacs, C. E. 2001. The antimicrobial function of milk lipids. pp. 271-285). Indeed, some free fatty acids (“FFA”) and monoacylglycerides present in abundance in bovine milk/whey exhibit antimicrobial activity against several pathogenic micro-organisms (Isaacs, 2001). Additionally, bovine milk/whey lipids contain non-negligeable amounts of bioactives components such as unsaturated fatty acids (UFA) regulating, even at low concentrations, diverse biological functions in living organisms (Jensen, R. G. 2002. The composition of bovine milk lipids: January 1995 to December 2000. Journal of Dairy Science 85: 295-350).

### SUMMARY OF THE INVENTION

One aspect of the present invention is to provide for a method for interfering with the hyphal growth of fungi by exposing the fungi to an antimicrobial agent derived from cream.

According to an embodiment of the invention, there is provided a method for interfering with the hyphal growth of C. albicans by exposing the C. albicans to antimicrobial agent(s) derived from cream.

In a particular case, the antimicrobial agents derived from cream which are used to interfere with the hyphal growth of fungi may be one or more of capric, lauric, myrsitoleic, linoleic, arachidonic and gamma-linolenic acids.

According to an embodiment of the invention, there is provided a method for interfering with hyphal growth of fungi by exposing the fungi to antimicrobial agent(s) derived from milk cream, whey cream, bovine whey cream and/or goat whey cream.

Another aspect of the invention provides a method for inhibiting the growth of microbes through the exposure of the microbes to an antimicrobial agent derived from cream. In particular, these microbes are fungal pathogens, such as C. albicans and A. fumigatus.

According to an embodiment of the invention, there is provided a method for inhibiting the growth of microbes through the exposure of the microbes to an antimicrobial agent derived from milk cream, whey cream, bovine whey cream and/or goat whey cream.

In a particular case, the antimicrobial agent derived from cream used to inhibit the growth of microbes C. albicans and A. fumigatus is an unsaturated free fatty acids enriched fraction of the cream. According to a another embodiment of the invention, the free fatty acids may be capric, lauroleic, 12-methyldodecanoic, myristoleic and/or gamma-linolenic acid.

According to another embodiment of the invention, there is provided a pharmaceutical composition comprising a free fatty acid in a pharmaceutically acceptable carrier. A further embodiment of the invention provides for a pharmaceutical composition comprising a free fatty acid in a pharmaceutically acceptable carrier for topical and/or systemic administration.

### BRIEF DESCRIPTION OF THE DRAWINGS

The embodiments of the present invention shall be more clearly understood with reference to the following description of the preferred embodiments and to the accompanying figures, in which:

FIG. 1 illustrates the antihyphal growth activity of total polar and neutral lipids enriched fractions derived from whey cream;

FIG. 2 illustrates the kinetics of germination inhibition of C. albicans by free fatty acids in different hyphae inducing conditions;

FIG. 3 illustrates microscopic observations of cells after 6 h incubation at 37° C.

FIG. 4 illustrates antifungal activity of unsaturated free fatty acids (“UFFA”) derived from bovine whey cream lipids; and

FIG. 5 illustrates the antifungal activity of UFFA derived high performance liquid chromatography (HPLC) fractions.

### DESCRIPTION OF THE INVENTION

### I. Materials and Methods

### Materials and reagents

Fresh unpasteurised whey cream was obtained from Saputo Inc. (Montreal, Canada). All lipid standards were purchased from Sigma-Aldrich, except for arachidonic and conjugated linolenic acids that were purchased from Matreya Inc. (Biolynx Inc, Ontario, Canada). The 11-methyldodecanoic acid was from Larodan Fine Chemical (Sweden). All other materials and solvents were of the highest purity or high-performance HPLC grade (Fisher Scientific).

### Instrumentation

HPLC analysis and purification were performed on a Beckman-Coulter HPLC Gold® system composed of two pumps, a module solvent (model 126), a UV spectrophotometric detector (model 168), a fractions collector (SC100) and a 500 μl sample loop injector (Reodyne 7725i). The recorded HPLC spectra were analysed using the 32 Karaf software (Beckman-Coulter). GC-MS analysis were performed by the Lipid Analysis Unit at the Scottish Crop Research Institute in Invergowrie, Dundee, Scotland. Gas chromatographic (GC) analysis was performed with an GC-FID 6809N Network System equipped with an Agilent 7683 Series Injector and an FID detector (Agilent Technology, Palo Alto, USA). The chemical duty pump used for SPE (Solid Phase Extraction) was from Millipore (Model WP6111560). Microscopic examination and photographs were done with a Axivert 135 TV inverted microscope (Zeiss).

### Extraction and Fractionation of Total Lipids

Total lipids of whey cream were extracted according to the Bligh-Dyer method (Bligh, E. G. and Dyer, W. J. 1959. A rapid method of total lipid extraction and purification. Can. J. Med. Sci. 37: 911-917). The total lipid fraction was separated into polar and neutral lipids by counter-current distribution (Galanos D S, Kapoulas V M (1962) Isolation of polar lipids from triglyceride mixtures. J Lipid Res 3: 134-137.) The yields of each fraction were determined gravimetrically. Typically, from 300 g of whey cream, about 150 g of total lipids were obtained whereas counter current distribution provided about 2 g and 130 g of polar and neutral lipids respectively. These three lipid fractions were assayed for activity.

FFAs were also purified following saponification of total lipids of whey cream as follow. Total lipids of fresh and unpasteurized bovine whey cream were extracted according to the Bligh-Dyer procedure (Bligh and Dyer, 1959). Total lipids (10 g) were then subjected to saponification for 60 min at 60° C. in a 1 L glass beaker containing 760 ml of ethanol (96%) and 16 g of potassium hydroxide. After cooling to room temperature, the mixture was filtered (40 μm) and acidified to pH 1 with 5 N HCl. The volume was adjusted to 1 L with water and FFA were extracted with hexane (4×400 ml). The extract (4×400 ml) was neutralized by washing with water and dried under nitrogen. FFAs were futher purified by solid phase extraction as describe above. The resulting FFAs were reconstitute at 20 mg/ml in 96% ethanol and assay for activity.

### Fractionation of Total Polar Lipids by Solid Phase Extraction

Polar lipids were separated into sterols/monoacylglycerol/diacylglycerols, phospholipid and free fatty acids fractions by Solid Phase Extraction as described previously (Kaluzny, M. A., Duncan, L. A., Merritt, M. V. and Epps, D. E. 1985. Rapid separation of lipid classes in high yield and purity using bonded phase columns. J. Lipid Res. 26: 135-140; Vaghela, M. N. and Kilara, A. 1995. A Rapid Method for Extraction of Total Lipids from Whey-Protein Concentrates and Separation of Lipid Classes with Solid-Phase Extraction. Journal of the American Oil Chemists Society 72: 1117-1121). Essentially, a Mega Bond Elut Flash (25 g) disposable aminopropyl column (Varian) was placed onto an Erlenmeyer flask connected to a chemical duty pump (Millipore). A vacuum of 10-12 kPa was constantly maintained during the procedure. Polar lipids (500 mg) dissolved in 20 ml of chloroform were applied under vacuum to the column which had been pre-washed twice with 100 ml portions of hexanes. Sterols/monoacylglycerols/diacylglycerols, FFAs and phospholipids were sequentially eluted with 225 ml of chloroform/2-propanol 2:1 (v/v), 225 ml of acetic acid/diethyl ether 1:50 (v/v) and 225 ml of methanol, respectively. Each eluate was dried under nitrogen and assayed for activity. The yield of each lipid fraction was determined gravimetrically. About 50 mg of free fatty acids, 120 mg of phospholipids and 270 mg of cholesterol, monoacylglycerols and diacylglycerols “enriched” fractions were typically obtained from 500 mg of polar lipids.

### Urea Fractionation

FFAs were fractionated into saturated free fatty acid (“SFFA”) and unsaturated free fatty acid (“UFFA”) enriched fractions by the urea inclusion procedure (Traitler, H., Willie, H. J., and Studer, A. 1988. Fractionation of Blackcurrant Seed Oil. Journal of the American Oil Chemists Society 65: 755-760). Essentially, methanol (12 ml) and urea (4 g) were added to dried FFA extract (1 g) placed into a small screw cap vial (20 ml). Under constant stirring, the mixture was heated to 75° C., or until it became clear, and then cooled slowly to 2° C. After completion of urea crystallization at 2° C. (15 h), the UFFA enriched methanol phase was separated from the urea crystals by centrifugation (5 min) at 1000×g. UFFA, as well as SFFA present in the urea crystal, were recovered as described by Traitler et al., (1988). Yields were determined gravimetrically. From this procedure, about 25 mg of UFFA and 900 mg of SFFA were obtained from approximately 1 g of FFA. FFA, UFFA and SFFA fractions were reconstituted at 20 mg/ml in 96% ethanol and 2 μl of binary ethanol dilutions were tested in quadruplicate for antifungal activity.

### Separation of Free Fatty Acids by HPLC

For the antigermination activity, the FFA enriched fraction was further fractionated by high-performance liquid chromatography (HPLC) on a semi-preparative silica-based normal phase column (Spherisorb S10W, 10 μm, 10×250 mm, Waters) using a binary solvent system: solvent A, hexane/tetrahydrofuran 97:3 (v/v); solvent B, hexane/tetrahydrofuran 98:2 (v/v). FFAs (˜20 mg in 500 μl of solvent B) were applied to the column pre-equilibrated in solvent A and eluted by a linear gradient to 100% B from 20 to 240 min at a flow rate of 1 ml/min. UV detection was used to monitor effluent at 210 nm. Collected fractions (4 ml) were dried under nitrogen and reconstituted in ethanol. The concentration of FFAs was determined enzymatically by colorimetric assays (Roche Diagnostic) using stearic acid as reference.

For antifungal activity, the UFFA enriched fraction was further separated by reverse-phase HPLC on a semi-preparative C18 column (Prep Nova-pak® HR C18, 6 μm, 60 Å, 7.8×300 mm, Waters) using a Beckman-Coulter-HPLC Gold® system. UFFA (about 4.5 mg) dissolved in 50% ethanol were applied to the column pre-equilibrated in 50% acetonitrile: 0.1% TFA and eluted by a linear gradient to 100% acetonitrile: 0.1% TFA from 0 to 70 min at a flow rate of 8 ml/min. UV detection was used to monitor effluent at 215 nm. Water (10 ml) was added to each of the 45 collected HPLC fractions, and they were then extracted three times with hexane (3×10 ml). After drying under nitrogen, each fraction was reconstituted in 30 μl of ethanol (70%) and 3 μl of each were tested in duplicate for antifungal activity. Antifungal assay using commercial FFA were done in quadruplicates.

### Esterification and Gas-Chromatography

FFA were dissolved in 1 ml of 0.5 N methanolic-HCL (Supelco) and heated at 50° C. for 10 min with occasionally hand shaking. After cooling to room temperature, water (1 ml) was added and the resulting fatty acid methyl esters (“FAME”) were extracted twice with n-pentane (2×1 ml). Organic phases were pooled and evaporated to dryness under nitrogen. FAME were dissolved in methylene chloride (0.02 ml) and transferred into small vials with glass inserts (Agilent Technology, Palo Alto, USA). The FAME were kept in the dark at −20° C. until analysis by gas chromatography.

Gas chromatographic (“GC”) analysis was performed with an GC-FID 6809N Network System equipped with an Agilent 7683 Series Injector and an FID detector (Agilent Technology, Palo Alto, USA). FFA were separated on a 30 m×0.32 mm ID capillary column coated with a 0.20 μm film of polyalkylene glycol (SPB-PUFA, Supelco). After holding the oven temperature at 50° C. for 2 min, the column was temperature-programmed at 4° C./min to 210° C. Helium was used as carrier at a velocity of 34 cm/sec. Individual FFA species were identified by comparison of retention times with those of known standards (i.e. 37 components FAME mix from Supelco or individual FFA derivatized as FAME).

### Mass Spectroscopy Analysis

Mass spectroscopy analysis was carried out in negative mode using a Micromass Quattro II triple Quadrupole Mass Spectrometer equipped with an electrospray source. Samples dissolved in 50% isopropanol containing 25 mM triethylamine were infused at a flow rate of 120 μl/h. Data were accumulated in MCA mode for one minute and analyses were carried out using MassLynx version 3.5 software. Nitrogen was used as curtain gas (400 l/h) and nebulising gas (20 l/h). The ESI capillary was set at 2.5 kV while the MS analysis was carried out at a cone voltage of 25 V, a scan rate of 300 Da/s with an inter-scan delay of 0.1 s and a scan range of 135-500 Da. The resolving power was set to obtain unit resolution.

### Analysis of Lipid Fractions by HPTLC

Lipid fractions derived from total whey cream lipids were evaluated for their purity by high-performance thin layer chromatography (HPTLC-HLF, 150 μm, 10×20 cm, Analtech). Typically, between 20 and 80 μg of each lipid fraction were spotted on HPTLC plates. Plates were developed vertically in a solvent system of hexane/diethyl ether/acetic acid 70:30:1 (v/v/v). Visualization of lipids was done by spraying plates with 40% H2SO4 (v/v) followed by 15 minutes incubation at 110° C.

### Germination Inhibition Assay

The Candida albicans SC5314 strain (Fonzi, W. A. and Irwin, M. Y. 1993. Isogenic strain construction and gene mapping in Candida albicans. Genetics 134: 717-728.) was used to investigate the antihyphal activity of whey cream lipids. This strain was routinely grown as yeast cells at 30° C. in YPD medium (1% yeast extract, 2% peptone, 2% dextrose). Germination was induced by incubating cells in dextrose-free Sabouraud (Difco; Joshi, K. R., Gavin, J. B., and Bremner, D. A. 1973. The formation of germ tubes by Candida albicans in various peptone media. Sabouraudia. 11: 259-262.) at 37° C. in 96-wells microtiter plates (Costar 3595). Briefly, cells from fresh YPD cultures were harvested and washed twice with sterile water. Washed cells were suspended in dextrose-free Sabouraud at a density of 5×103 cells/ml and then immediately distributed to microtiter wells containing an equal volume of dextrose-free Sabouraud supplemented with different concentrations of lipids. After 15 h incubation at 37° C. in an atmospheric incubator without agitation, cells were washed and fixed with a fresh 1% formaldehyde solution. The ability of lipids to inhibit germination was determined by microscopic observations. The minimal inhibitory concentration (MIC) was defined as the lowest concentration of lipids that completely inhibited the germination of C. albicans after 15 h at 37° C. All lipids were dissolved in ethanol and no more than 1% ethanol (final concentration) was used in the incubating medium.

### C. albicans Beta-Galactosidase and XTT Assays

Candida albicans ZK3379 strain (CAI-4 HWP1-lacZ; Hogan, D A, Vik A, Kolter R (2004). A Pseudomonas aeruginosa quorum-sensing molecule influences Candida albicans morphology. Mol Microbiol 54: 1212-1223.) was used for beta-galactosidase and XTT assays. Cells from this strain were grown in YPDA at 30° C. to a density of about 1×10⁸ cells/ml (hemacytometer). These late exponentially growing cells were then washed twice with water and kept on ice until used. Germination was induced by incubating cells (final density of 5×10⁵ cells/ ml) at 37° C. in 2.5 ml of either dextrose-free Sabouraud (Joshi et al., 1973), Lee's (Lee K L, Buckley H R, Campbell C C (1975). An amino acid liquid synthetic medium for the development of mycelial and yeast forms of Candida albicans. Sabouraudia 13: 148-153, Spider (Liu H, Kohler J, Fink Gr (1994). Suppression of hyphal formation in Candida albicans by mutation of a STE12 homolog. Science 266: 1723-1726 or Hypha-Forming Media (Biswas S K, Yokoyama K, Kamei K, Nishimur K, Miyaji M (2001) Inhibition of hyphal growth of Candida albicans by activated lansoprazole, a novel benzimidazole proton pump inhibitor Med Mycol 39: 283-285.), supplemented with FFAs. All lipids were dissolved in ethanol. Assays were performed in duplicate in 24-wells mictotiter plates (Costar 3526) without agitation. After appropriate incubation times (i.e. 0, 2, 4 or 6 h), microtiter plates were centrifuged for 5 min at 2000 rpm (Jouan inc. CR3i) and supernatents, discarded. XTT assays were done immediately and essentially as described (Honraet K, Goetghebeur E, Nelis H J (2005). Comparison of three assays for the quantification of Candida biomass in suspension and CDC reactor grown biofilms. J Microbiol Methods 63: 287-295). For beta-galactosidase assays, microtiter plates were frozen at −80° C. and processed later as described (Kippert F (1995). A rapid permeabilization procedure for accurate quantitative determination of beta-galactosidase activity in yeast cells. FEMS Microbiol Lett 128: 201-206; Hogan et al., 2004) but directly in microtiters plates. Absorbances were measured in 96-well microtiters plates using a microtiter plate reader (Microplate Reader MR600), equipped with filters of 410 nm (beta-galactosidase) and 490 nm (XTT), respectively.

### Antifungal Assays

The antifungal activity was evaluated in 96-well microtiter plates (Costar 3595) using Sabouraud as incubating medium for A. fumigatus and dextrose-free Sabouraud for C. albicans. Spores of A. fumigatus (Jeanine Joly, Université de Montréal) were germinated at 25° C. on slant agar Sabouraud media. They were harvested by washing vigorously slant cultures with 5 ml of 0.9% NaCl. Coarse debris were removed by filtrating the Aspergillus spores suspension through a sterile cotton plugs inserted into a Pasteur pipet. Monodisperse spores suspensions were obtained following brief sonications in a water bath sonicator (Branson 1210). Spores of A. fumigatus were adjusted at 1×10⁴/ml in Sabouraud liquid media using an hemacymeter. Blastospores of the C. albicans SC5314 strain were prepared as for germination inhibition assays (see above). Microtiter wells, containing 0.1 ml of incubating media supplemented with different concentrations of FFA, were inoculated with 0.1 ml of either A. fumigatus or C. albicans spore suspensions. Trays were incubated at 30° C. for A. fumigatus or at 37° C. for C. albicans in atmospheric incubators for 40 h. The minimal inhibitory concentration (IC₅₀) was defined as the lowest concentration reducing by 50% the optical density at 630 nm (Microplate Reader MR600, Dynatech) of samples to sample-free control (i.e. 1.05% ethanol). Wells were washed three times with Sabouraud before optical reading.

### Candida albicans Filamentous Growth Inhibited by Gamma-Linolenic Acid

The effects of fatty acids gamma-linolenic acid and alpha-linolenic acid on filamentous growth of Candida albicans were determined in a filamentation assay. C. albicans reporter strain ZK3379 (HWP1-lacZ; Hogan et al., 2004) was propagated in yeast extract-peptone-dextrose (YPD) for 24 h at 30° C. Prior to the assay, cells were washed twice with phosphate-buffered saline (PBS 1×). Flasks (125 ml) containing 10 ml of prewarmed RPMI 1640/20 mM MOPS (pH 7.0) were inoculated with washed cells at a final concentration of 10⁶ cells per ml. Working solutions of fatty acids (25 mM and 6.25 mM) were prepared freshly in ethanol 70%. Fatty acids were diluted in medium at final concentrations of 100 μM and 25 μM. An equal volume (40 μl) of ethanol 70% was added to control cultures. In all cultures, the final concentration of ethanol was 0.4% (v/v). Flasks were incubated at 37° C. for 18 h with shaking (ca. 200 RPM). Cells were collected by filtration (Millipore, 0.45 μm) and washed with PBS 1×. beta-galactosidase was assayed according to the method of Rose and Botstein (1983), where crude protein extracts are prepared and the activity is normalized to the amount of protein assayed. Relative amounts of beta-galactosidase activity were used as a measure of the amount of filamentous growth. The activity was compared with that in the control culture.

### Cytotoxicity Assays

Cytotoxicity was evaluated using Peripheral Blood Mononuclear cells (PBMC) of volunteers donors (Royal Victoria Hospital, Montreal). These cells were purified by the ficoll-Paque PLUS according to the manufacturer (Amersham-Bioscience). PBMC were used at a density of 100 000 cells/ml in RPMI 1640 (Sigma) supplemented with 10% heat-inactivated fetal bovine serum, 0.3 g/L of L-glutamine, 2 g/L sodium carbonate, 100 U/ml of penicillin and 100 μg/ml of streptomycin (Sigma). Assays were done in 96-well microtiter plates (Costar 3595) and the cytotoxicity was evaluated after 24 hours of incubation at 37° C. in a 5% CO₂ atmospheric incubator using the LDH Cytotoxicity detection kit, according to the manufacturer (Roche Diagnostic). FFA concentration able to kill 50% of PBMC relative to a sample-free control was defined as the toxic concentration (TC₅₀). FFA were diluted in ethanol (96%) and no more than 1% ethanol (final concentration) was used in assays. Ethanol (1%) was used as negative control.

### II. Results and Discussion

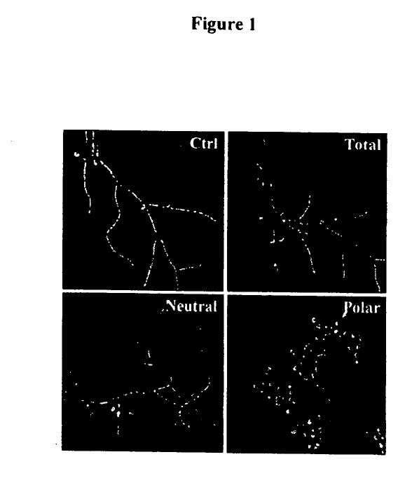

The Ctrl panel of FIG. 1 illustrates that C. albicans readily germinated and formed hyphae in dextrose-free Sabouraud medium when incubated at 37° C. for 15 h. When polar lipids were present at a concentration of 100 μg/ml, the germination of the C. albicans was totally inhibited (see FIG. 1 Polar panel). Minimal inhibitory concentrations (MIC), which represent the lowest concentration of lipids that completely inhibited the germination of C. albicans, are shown in Table 1. The MIC for the polar lipid enriched fraction was found to be about 100 μg/ml whereas those values were beyond 500 μg/ml for the total, and neutral lipid, enriched fractions (Table 1). Such activity for the polar lipid enriched fraction is not restricted to whey cream as polar lipids derived from milk cream are also active at inhibiting the germination of C. albicans (data not shown). In addition, the polar lipid enriched fraction derived from whey cream is also active in Lee's (Lee et al., 1975) and Hypha-Forming Media (Biswas et al., 2001), indicating that this inhibiting activity is not restricted to dextrose-free Sabouraud medium (data not shown). It is important to note that the polar lipid enriched fraction does not significantly affect the growth of C. albicans under its yeast form, demonstrating that the effect is specific to the germination of this pathogen (data not shown). HPTLC analysis shows that the polar lipid fraction is enriched in phospholipids, free fatty acids, sterols, monoacylglycerols and diacylglycerols as compared to neutral and total lipid fractions which mainly contain triacylglycerols (data not shown).

### Fractionation of Polar Lipids

Polar lipids were separated into three fractions that were either enriched in (1) phospholipids, (2) free fatty acids or (3) sterols, monoacylglycerols and diacylglycerols, as determined by HPTLC analysis (data not shown). These fractions were tested for their ability to inhibit germination of C. albicans. As shown in Table 1, the free fatty acid enriched fraction exhibited the strongest activity with a MIC of about 11 g/ml. The phospholipid enriched fraction also displayed activity, but it was weak as small hyphae could still be observed when phospholipids were used at concentrations as high as 500 μg/ml (data not shown). This activity could possibly be due to the presence of lyso-phosphatidylcholine as reported previously (Min J, Lee Y J, Kim Y A, Park H S, Han S Y, Jhon Gj, Choi W (2001). Lysophosphatidylcholine derived from deer antler extract suppresses hyphal transition in Candida albicans through MAP kinase pathway. Biochim Biophys Acta 1531: 77-89. The fraction enriched in sterols, monoacylglycerols and diacylglycerols did not affect the germination of C. albicans at concentrations below 500 μg/ ml (Table 1).

### Separation of Free Fatty Acids by HPLC

The pH of the dextrose-free Sabouraud medium was around 6.9 and was not found to be significantly affected by either 10 or 100 μg/ml of the free fatty acids fractions (data not shown). This suggested that a change in the assay pH was not likely to be the factor responsible of the activity of the free fatty acids enriched fractions. Therefore, the free fatty acid fraction derived from whey cream was further fractionated by HPLC on a semi-preparative silica-based normal phase column. Fifty-five fractions were collected and the amount of free fatty acids in each fraction was determined by the more sensitive enzymatic colorimetric test. Nevertheless, on the basis of this enzymatic method, which relies on the acid group, the MIC of unfractionated free fatty acids was found to be similar (14 μg/ ml, Table 1) to the MIC determined on the basis of the gravimetrical method reported in Table 1 (11 μg/ml). When testing HPLC fractions for their activity, fraction 16 was found to be the most active at inhibiting C. albicans germination. The MIC of this fraction was about 4 μg/ml (Table 1).

### Fatty Acid Composition of Active Fractions

GC-MS analysis reveals that the fraction 16 is still a mixture of free fatty acids, but contains less saturated and monounsaturated free fatty acids as compared to the unfractionated free fatty acids fraction (Table 2). Indeed, while the latter contains respectively 42.8% and 34.7% of saturated and monounsaturated free fatty acids, these values for fraction 16 are only 21.1% (Table 2). However, fraction 16 is enriched in PUFA, particularly in arachidonic (C20:4n-6; 4%) and linoleic acids (C18:2; 8.5%), which represent 12.5% of the fraction (Table 2). The unfractionated free fatty acids contain only 1.5% of PUFA (i.e. 0.2% of arachidonic and 1.3% of linoleic acids; Table 2). It was impossible to quantify linoleic acids isomers by GC-MS analysis, although linoleic acid (C18:2n-6) and a small amount of CLA was detected in fraction 16 as well as in the unfractionated free fatty acids fraction (data not shown). When comparing each of the free fatty acids, more myristoleic (C14:1n-5), pentadecanoic (C15:0), linolenic (C18:2) and arachidonic (C20:4n-6) acids were found in fraction 16 as compared to the unfractionated free fatty acids (Table 2). Thus, as fraction 16 is about 3-4 fold more active than the unfractionated free fatty acid fraction, this suggests that myristoleic, pentadecanoic, linoleic and arachidonic acids could be responsible for its greater activity.

### Biological Activity of Individual Free Fatty Acids

In order to determine if free fatty acids present in fraction 16 are individually active at inhibiting the induced hyphal growth of C. albicans, commercial preparations for each free fatty acid present in fraction 16 were assayed. The most abundant isomers of linoleic acid (i.e. CLA (C18:2 9c-11t) and C18:2n-6) present in bovine milk lipids were used. As expected, stearic (C18:0), heptadecanoic (C17:0), palmitic (C16:0), pentadecanoic (C15:0) and myristic (C14:0) acids, which are less abundant in fraction 16, are inactive at inhibiting C. albicans germination, even at the highest concentration used (i.e. 81 μM; Table 3). Thus, even though pentadecanoic acid is more abundant in fraction 16, this indicates that this free fatty acid, as well as the other saturated free fatty acids, is not responsible of the activity of free fatty acids derived from whey cream. However, we found that lauric acid (C12:0), present at similar level in both fractions, displays activity with a MIC of 9 μM (Table 3). Even if not present in our fractions, capric acid (C10:0) was found to be similarly active to lauric acid (Table 3). This is particularly interesting since, as compared to other saturated fatty acids, lauric and capric acids were previously found to be highly active at killing C. albicans yeast cells although at a quite elevated concentration of 10 mM (Bergsson, G., Arnfinnsson, J., Steingrimsson, O., and Thormar, H. 2001. In vitro killing of Candida albicans by fatty acids and monoglycerides. Antimicrob. Agents Chemother. 45: 3209-3212). As a thousand fold less concentrated solution of lauric or capric acid is not toxic for mammalian cells in vitro, this raises the possibility that these two saturated fatty acids could be used in vivo for the treatment of invasive fungal infections (Bergsson et al., 2001; data not shown).

The majority of unsaturated free fatty acids used displayed inhibition activity, regardless of their length or their degree of unsaturation (Table 3). Hyphal development of C. albicans is completely inhibited when they are used at concentrations varying between 9 and 81 μM, depending on the free fatty acid. Myristoleic, palmitoleic and conjugated linoleic acids (CLAs) were found to be the most active fatty acids inhibiting C. albicans germination with MICs of 9 μM (Table 3). Arachidonic and linoleic acids are also active at inhibiting the germination of C. albicans in vitro with MIC of 27 and 81 μM respectively (Table 3). Finally, no or weak activity was found for oleic (C18:1n-9) and vaccenic (C18:1n-7) acids (Table 3). The latter could be expected since fraction 16 is more active than the unfractionated free fatty acids and these two monounsaturated fatty acids are less abundant in fraction 16 (Table 2). None of the active free fatty acid altered the pH of the culture medium (data not shown). Therefore, the germination inhibition activity of free fatty acids derived from whey cream could be mainly attributed to lauric (C12:0), myristoleic (C14:1n-5), palmitoleic (C16:1n-7), linoleic (C18:2n-6) and arachidonic (C20:4n-6) acids. Indeed, fraction 16, which is 3-4 fold more active than the unfractionated free fatty acids, contains about 3 times more active free fatty acids (Table 1 and Table 2). Kinetic of germination and inhibition of different hyphae inducing media

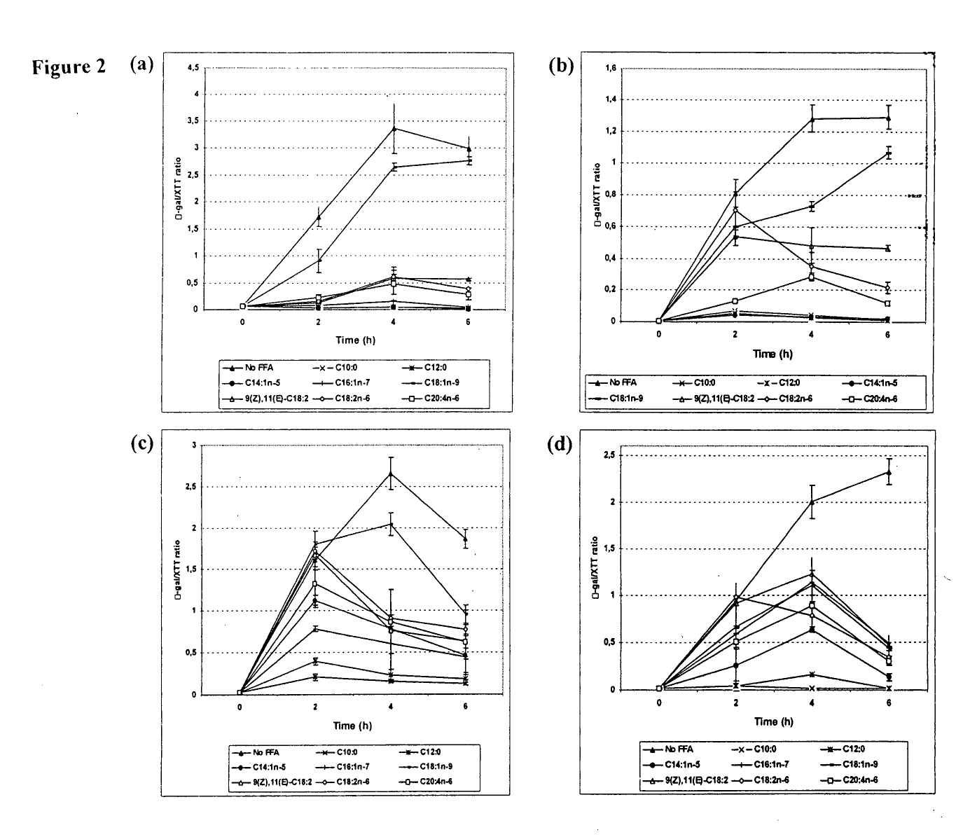

As inhibition of germination of C. albicans by molecules could be the result of early and/or late blocking events, a more comprehensive analysis of the activity of free fatty acids was performed using multiple time points and other germ tube inducing media. Therefore, germination was monitored at 0, 2, 4 and 6 h in dextrose-free Sabouraud, Lee's, Spider and HFM7 (i.e. serum) media using the C. albicans HWP1-lacZ assay: beta-galactosidase measurements should reflect the morphological change from yeast cells to hyphal growth (Hogan et al., 2004). beta-galactosidase results were normalized using the XTT assay (Honraet et al., 2005) to correct for biomass increase. Microscopic observations of cells after 6 h incubation are also presented for each inducing conditions and correlated with beta-galactosidase data (FIG. 2).

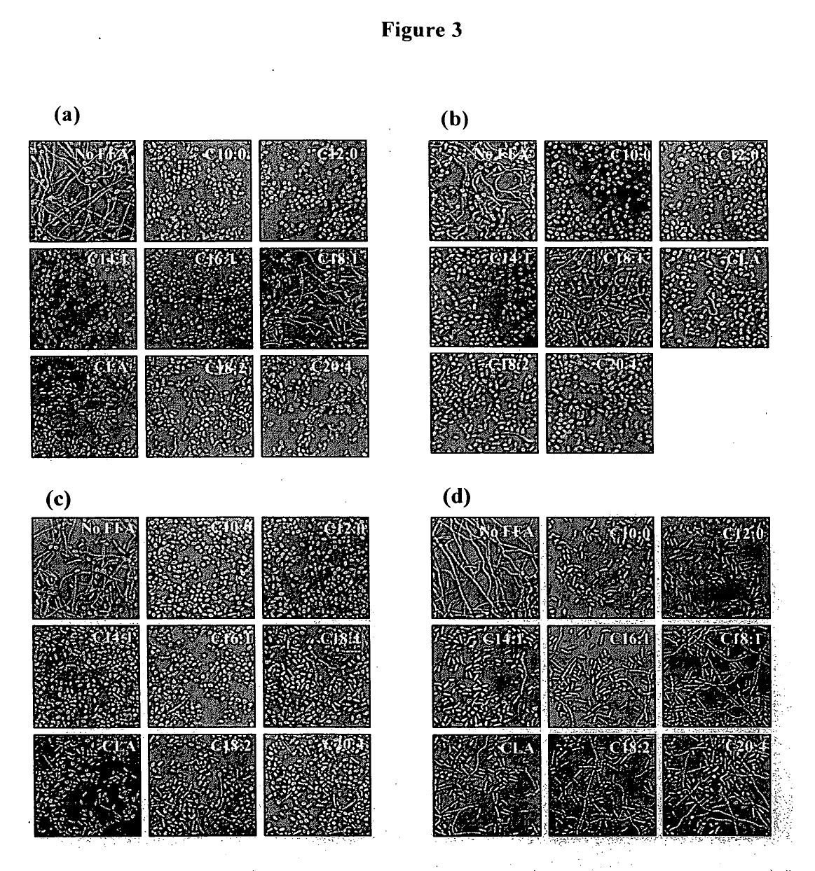

Regardless the inducing media used, cells incubated in the absence of free fatty acids germinated well and formed hyphae, but progressively reverted to yeast mode of growth after 4-6 h (FIG. 2, No FFA). This was probably due to an inoculum size effect, since we used an inoculum of 5×10⁵ cells/ml and the yeast to mycelium transition is blocked by quorum sensing molecules (i.e. farnesol) when cells density become higher than 10⁶ cells/ml (Hornby J M, Jensen E C, Lisec A D, Tasto J J, Jahnke B, Shoemaker R, Dussault P, Nickerson K W (2001). Quorum sensing in the dimorphic fungus Candida albicans is mediated by farnesol. Appl Environ Microbiol 67: 2982-2992). Analysis of the activity of free fatty acids revealed that some exhibited inducing medium dependent effects, but general trends could be observed. Indeed, capric (C10:0) and lauric (C12:0) acids were found to be the most active fatty acids and completely inhibited the emergence of germ tubes in all inducing conditions tested (FIG. 2). On the other hand, blastospores incubated in the presence of conjugated linoleic or linoleic acids produced germ tubes (2-4 h), but their further elongation was blocked (FIG. 2, 4-6 h). This is particularly evident using Lee's, Spider and HFM7 inducing media (FIG. 2b, c, d). Finally, the activity of myristoleic (C14:1n-5), palmitoleic (C16:1n-9), oleic (C18:1n-9) and arachidonic (C20:4n-6) acids seemed to be inducing media dependent. Indeed, oleic acid exhibited a weak or no activity against the germination of C. albicans in all inducing media but HFM7 (FIG. 2). In Spider and HFM7 media, myristoleic, palmitoleic and arachidonic acids partially inhibited the appearance of germ tubes and also block their elongation to form hyphae (FIG. 2c, d). In contrast, myristoleic (C14:1n-5) and arachidonic (C20:4n-6) acids strongly inhibited the appearance of germ tubes in dextrose-free Sabouraud and Lee's inducing media (FIG. 3b). It was impossible to evaluate the effect of palmitoleic acid (C16:1n-7) in Lee's medium as this unsaturated free fatty acids was toxic for C. albicans blastospores in this condition (data not shown). Microscopic observations of cells after 6 h incubation indicated that all the beta-galactosidase data correlate with cellular morphology (FIG. 3). However, this correlation was less obvious for some free fatty acids in HFM7 medium, suggesting that changes in cellular morphology due to a reduced activity of the HWP1 promoter could be delayed in this condition (FIG. 3d).

### Antifungal Activity of FFA Derived from Bovine Whey Cream Lipids

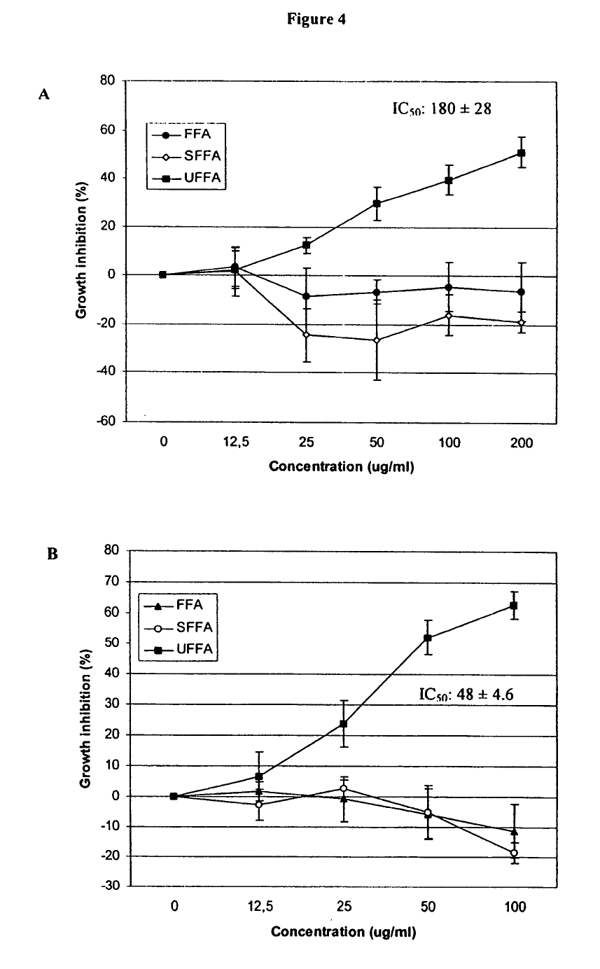

FFA originating from direct saponification of bovine whey cream lipids were assayed against the germination of C. albicans in vitro in dextrose-free Sabouraud. Results presented in Table 1 demonstrate that these FFA can completely inhibit in vitro the germination of C. albicans at about 33 μg/ml (MIC, Table 4). While FFA from bovine whey cream are active at inhibiting the germination of C. albicans, this activity seems to be specific to the hyphal development of C. albicans (data not shown). A. fumigatus, is an emerging fungal pathogen that grows exclusively as hyphae. The results presented in FIG. 4A indicate that even if bovine whey FFA were active at inhibiting the germination of C. albicans in vitro, they were inactive against A. fumigatus (FIG. 4A). Indeed, growth of A. fumigatus seemed unaffected even at a concentration as high as 200 μg/ml (FIG. 4A). However, when these FFA were fractionated by the urea inclusion procedure, the resulting unsaturated FFA enriched fraction (UFFA) was found to exhibit, in a dose-dependent manner, a significant antifungal activity against A. fumigatus. Indeed, UFFA can inhibit 50% A. fumigatus growth at a concentration of 180 μg/ml (FIG. 4A). No such activity was observed with the FFA fraction enriched in saturated free fatty acids (SFFA, FIG. 4A).

The UFFA enriched fraction was also found to be active in vitro against C. albicans. Indeed, we observed that at low concentrations, UFFA were highly active at inhibiting the induced hyphal growth of C. albicans. While unfractionated FFA (FFA) inhibit completely the induced hyphal growth of C. albicans at 33.3 μg/ml, the UFFA enriched fraction did the same at 3.7 μg/ml (Table 4). In addition to this activity, we also found that UFFA strongly inhibited the development of C. albicans, irrespectively of its growing form (FIG. 4B). Using dose response curves, the UFFA concentration required to inhibit by 50% the growth of C. albicans was approximately 48 μg/ml (FIG. 4B). In contrast to UFFA, SFFA were either inactive or slightly stimulated the growth of C. albicans at elevated concentrations (FIG. 4B). Additionally, as compared to unfractionated FFA and UFFA, SFFA were less active against the induced hyphal growth of C. albicans, inhibiting completely the germination of C. albicans at 100 μg/ml (Table 4).

### Isolation and Identification of Antifungal Compounds

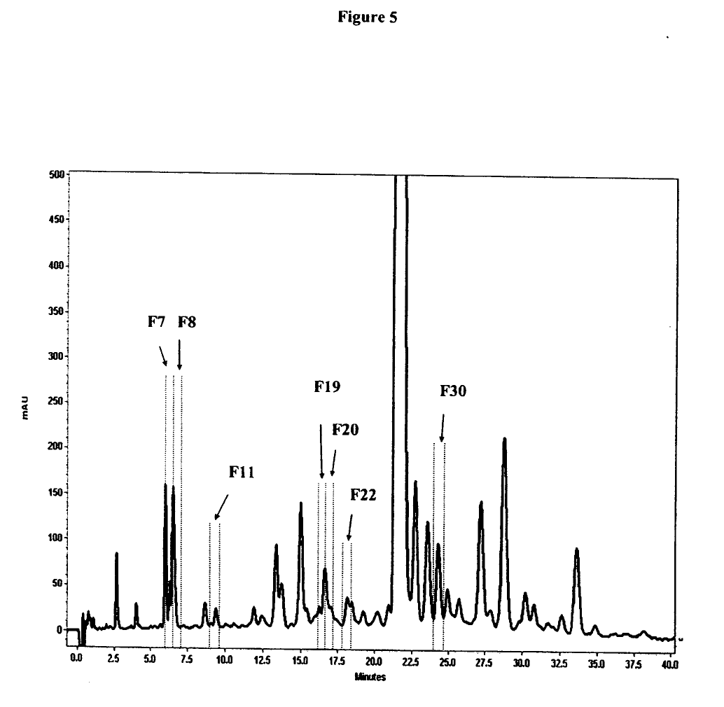

The antifungal activity of UFFA against both A. fumigatus and C. albicans was further investigated using an antifungal assay-guided HPLC fractionation. From this procedure, five fractions (F7, F8, F11, F19 and F20) exhibiting an antifungal activity against both A. fumigatus and C. albicans were identified. Additionally, two others fractions (F22 and F30) were found to be active against C. albicans specifically. The HPLC chromatogram of the UFFA enriched fraction is shown in FIG. 5. Methyl ester derivatives of components present in active fractions were analysed using a capillary gas chromatography. According to a reference FAME mixture, the antifungal components of each fraction were identified as capric acid (C10:0; F7 and F8), lauroleic acid (C12:1; F11), myristoleic acid (C14:1n-5; F19 and F20), 12-methyldodecanoic acid (iso-C13:0; F22) and gamma-linolenic acid (C18:3n-6; F30). The identity of these FFA was further confirmed using mass spectroscopy and HPLC analysis with commercial reference compounds (data not shown).

Quantitative assays were performed using commercial preparations of identified FFA as none were isolated in amounts sufficient to perform dose-response experiments. Lauroleic acid was excluded from these assays as the isomer present in bovine milk (C12:1n-3) was not commercially available. As expected, all of the identified FFA exhibited an in vitro antifungal activity, with C. albicans being generally more susceptible than A. fumigatus, except for capric acid (Table 5). Capric and myristoleic acids inhibited by 50% the growth of A. fumigatus at 127 and 192 μM respectively. These two FFA were also active against C. albicans with IC₅₀ of 182 and 82 μM respectively (Table 5). The gamma-linolenic acid, which was initially isolated by its ability to inhibit specifically the growth of C. albicans, was found to be active against both C. albicans and A. fumigatus in vitro (Table 5). The gamma-linoleinc acid was the FFA exhibiting the highest antifungal activity with IC₅₀ of 2.34 and 13.4 μM against C. albicans and A. fumigatus respectively (Table 5). Similarly, the 12-methyldodecanoic acid was active against both C. albicans and A. fumigatus, even if it was isolated by its ability to inhibit specifically the growth of C. albicans. As shown in Table 5, about 49 and 283 μM of 12-methyldodecanoic acid were required to inhibit 50% of C. albicans and A. fumigatus growth respectively (Table 5).

Further studies on the antifungal activity of gamma-linolenic acid using the RPMI 1640/20 mM MOPS (pH 7.0) as germ tube inducing media were conducted. Whereas this fatty acid exhibited antifungal activity in dextrose-free Sabouraud, such activity in RPMI 1640/20 mM MOPS media was not detected, even at a concentration of 100 μM (data not shown). However, gamma-linolenic acid was found to be active at inhibiting the germination of C. albicans, as monitored by the C. albicans HWP1-LacZ strain (Table 6; Hogan et al., 2004). Indeed, the germination of C. albicans seems to be completely inhibited with 100 μM of gamma-linolenic acid (Table 6).

### Cytotoxicity of FFA

To determine if the FFA identified above are toxic to humans cells, the FFA were assayed in vitro on Peripheral Mononuclear Cells (PBMC) using the LDH calorimetric method (Roche Diagnostic). This method measures the release of lactate dehydrogenase (LDH) from the cytosol of damaged cells into the supernatant (Roche Diagnostic). As shown in Table 5, the less toxic FFA was capric acid with a TC₅₀ of about 1.4 mM. Myristoleic, 12-methyldodecanoic and gamma-linolenic acids exhibited higher toxicity as compared to capric acid. Indeed, these three fatty acids killed 50% of PBMC at concentrations of 72, 70 and 41 μM respectively (Table 5). Interestingly, while all of the identified FFA exhibited a level of cytotoxicity in vitro, the antifungal activity of both capric and gamma-linolenic acids occurred at lower concentrations (Table 5).

### III. Conclusion

Semi-purified lipids fractions derived from whey cream are active in vitro at inhibiting the development of the pathogenic associated filamentous form of C. albicans. More precisely, this activity seems to be concentrated in unsaturated free fatty acids enriched fractions containing myristoleic, palmitoleic, linoleic and arachidonic acids, as well as contaminating saturated capric and lauric acids. Also demonstrated was that a fraction enriched in non-saturated free fatty acids such as lauroleic, myristoleic, 12-methyldodecanoic, palmitoleic and gamma-linolenic acids, as well as contaminating saturate capric acid, exhibit antifungal activity against both C. albicans and A. fumigatus. Whereas most of these free fatty acids represent only a small fraction of the total fatty acids present in dairy products, many of them are biologically active at low concentrations.

Considering the large quantity of milk processed each year by cheese making producers, whey could become a non-limiting source of these molecules for pharmaceutical compositions. In addition, the low concentration of the free fatty acids required for biological activity, readily allows for pharmaceutical compositions to be prepared comprising antimicrobial effective amounts of the free fatty acids in non-toxic concentrations. Medicinal topical creams could be prepared with the free fatty acids to inhibit antimicrobial growth. More specifically, topical creams containing the free fatty acids as a medicinal ingredient would be of use in cases such as a C. albicans infection.

TABLE 1
Antihyphal Growth Activity of Lipids Fractions Prepared from Whey
Cream.

| Lipids Enriched Fraction | MIC* (μg/ml) |
|---|---|
| Totals | >500 |
| Neutrals | >500 |
| Polars | 100 |
| Phopholipids | >500 |
| Sterols/MAG/DAG | >500 |
| Free fatty acids* | 11 |
| Free fatty acids^{§} | 14 |
| Fraction 16^{§} | 4 |

*Based on gravimetrical quantification method.
^{§}Based on enzymatic quantification method.

TABLE 2
Composition (%) of free fatty acids fractions derived from whey cream.

| Fatty acids | Unfractionated | Fraction 16* |
|---|---|---|
| Saturates | 42.8 | 21.1 |
| 12:0 | 3.1 | 3.3 |
| 14:0 | 9.0 | 6.0 |
| 15:0 | 1.0 | 4.0 |
| 16:0 | 21.5 | 7.2 |
| 17:0 | 0.5 | — |
| 18:0 | 7.7 | 0.6 |
| Monounsaturates | 34.7 | 21.1 |
| 14:1(n-5) | 1.3 | 3.1 |
| 18:1(n-9) | 24.2 | 11.4 |
| 18:1(n-7) | 7.6 | 6.6 |
| Polyunsaturates | 1.5 | 12.5 |
| 18:2 | 1.3 | 8.5 |
| 20:4(n-6) | 0.2 | 4.0 |

*From HPLC separation

TABLE 3
Antihyphal Growth Activity of Individual Free Fatty Acids.

| Fatty acids | *MIC μM (μg/ml) |
|---|---|
| Saturates | |
| 10:0 | 9 (1.55) |
| 12:0 | 9 (1.80) |
| 14:0 | >81 (18.5) |
| 15:0 | >81 (19.6) |
| 16:0 | >81 (20.8) |
| 17:0 | >81 (21.9) |
| 18:0 | >81 (23) |
| Monounsaturates | |
| 14:1(n-5) | 9 (2.04) |
| 16:1(n-7) | 9 (2.29) |
| 18:1(n-9) | >81 (18.5) |
| 18:1(n-7) | >81 (18.5) |
| Polyunsaturates | |
| 18:2(n-6) | 81 (22.7) |
| 18:2(CLA 9c-11t) | 9 (2.52) |
| 20:4(n-6) | 27 (8.22) |

*Based on gravimetrical quantification method.

TABLE 4
Anti-hyphal activity of FFA from bovine whey cream lipids

| Compound | MIC (μg/ml) |
|---|---|
| Unsaponificated FFA | 11.1 |
| Saponificated FFA | 33.3 |
| SFFA^{a} | 100 |
| UFFA^{b} | 3.7 |

^{a}SFFA: Saturated FFA
^{b}UFFA: Unsaturated FFA

TABLE 5
Antifungal activities and cytotoxicity of FFA

| | Antifungal activity | (IC₅₀, μM) | Toxicity (TC₅₀, μM) |
|---|---|---|---|
| Compound | A. fumigatus | C. albicans | PBMCs |
| Capric acid (C10:0) | 127 ± 11.3 | 182 ± 6.1 | 1428 ± 32 |
| Lauroleic acid (C12:1n?)^{a} | — | — | — |
| Myristoleic acid (C14:1n-5) | 192 ± 16.9 | 81.9 ± 9.2 | 71.9 ± 6.2 |
| 12-methyldodecanoic acid (C13:0 i) | 283 ± 42.9 | 49 ± 1.7 | 70 ± 3.6 |
| gamma-linolenic acid (C18:3n-6) | 13.4 ± 0.36 | 2.34 ± 1.40 | 41 ± 0.2 |

^{a}Not determinated

TABLE 6
Effect of gamma-linolenic acid on C. albicans filamentous growth
Relative beta-galactosidase expression (%)^{a}

| Dose (μM) | GLA |
|---|---|
| 25 | 5.9 (±6.8) |
| 100 | 0.5 (±0.7) |

^{a}Relative beta-galactosidase expression is expressed as a percentage of that of control cultures incubated in the absence of fatty acids. The results are means ± standard errors of one experiment carried out in duplicates.

1. A method for interfering with the hyphal growth of fungi comprising exposing the fungi to an antimicrobial agent derived from cream, wherein the antimicrobial agent is at a concentration sufficient to interfere with the hypal growth of fungi without preventing multiplication of the fungi.

2. The method according to claim 1, wherein the fungi is a human fungal pathogen.

3. The method according to claim 2, wherein the pathogen is Candida albicans.

4. The method according to claim 1, wherein the cream is milk cream.

5. The method according to claim 1, wherein the cream is whey cream.

6. The method according to claim 1, wherein the whey cream is bovine whey cream.

7. The method according to claim 15, wherein the whey cream is goat whey cream.

8. The method according to claim 1, wherein the antimicrobial agent is a polar lipids enriched fraction.

9. (canceled)

10. The method according to claim 8, wherein the polar lipids enriched fraction is a free fatty acids enriched fraction.

11. The method according to claim 10, wherein the concentration of free fatty acids enriched fraction is greater than about 11 micrograms/ml.

12. The method according to claim 10, wherein the free fatty acids enriched fraction is an unsaturated free fatty acids enriched fraction.

13. The method according to claim 12, wherein the concentration of the unsaturated free fatty acids enriched fraction is greater than about 3.7 micrograms/ml.

14. The method according to claim 10, wherein the free fatty acids enriched fraction is a saturated free fatty acids enriched fraction.

15. The method according to claim 14, wherein the concentration of the saturated free fatty acid enriched fraction is greater than about 100 micrograms/ml.

16. The method according to claim 10, wherein the free fatty acids include capric acid.

17. The method according to claim 16, wherein the concentration of the capric acid is greater than about 1.55 micrograms/ml.

18. The method according to claim 10, wherein the free fatty acids include lauric acid.

19. The method according to claim 18, wherein the concentration of the lauric acid is greater than about 1.8 micrograms/ml.

20. The method according to claim 10, wherein the free fatty acids include myristoleic acid.

21. The method according to claim 20, wherein the concentration of the myristoleic acid is greater than about 2.4 micrograms/ml.

22. The method according to claim 10, wherein the free fatty acids include palmitoleic acid.

23. The method according to claim 22, wherein the concentration of the palmitoleic acid is greater than about 2.29 micrograms/ml.

24. The method according to claim 10, wherein the free fatty acids include linoleic acid.

25. The method according to claim 24, wherein the concentration of the linoleic acid is greater than about 22.7 micrograms/ml.

26. The method according to claim 10, wherein the free fatty acids include conjugated linoleic acid.

27. The method according to claim 26, wherein the concentration of the conjugated linoleic acid is greater than about 2.52 micrograms/ml.

28. The method according to claim 10, wherein the free fatty acids include arachidonic acid.

29. The method according to claim 28, wherein the concentration of the arachidonic acid is greater than about 8.22 micrograms/ml.

30. The method according to claim 10, wherein the free fatty acids include gamma-linolenic acid.

31. The method according to claim 30, wherein the concentration of the gamma-linolenic acid is greater than about 25 micrograms/ml.

32. A method for inhibiting the growth of microbes comprising exposing the microbes to an antimicrobial agent derived from cream, wherein the antimicrobial agent is at a concentration sufficient to inhibit the growth of the microbes.

33. The method according to claim 32, wherein the microbes are fungal pathogens.

34. The method according to claim 33, wherein pathogens include Candida albicans or Aspergillus fumigatus.

35. The method according to claim 32, wherein the cream is milk cream.

36. The method according to claim 32, wherein the cream is whey cream.

37. The method according to claim 36, wherein the whey cream is bovine whey cream.

38. The method according to claim 36, wherein the whey cream is goat whey cream.

39. The method according to claim 32, wherein the antimicrobial agent is an unsaturated free fatty acids enriched fraction.

40. The method according to claim 32, wherein the free fatty acids includes capric acid.

41. The method according to claim 40, wherein the concentration of the capric acid is greater than about 127 micrograms/ml.

42. The method according to claim 39, wherein the free fatty acids includes lauroleic acid.

43. The method according to claim 39, wherein the free fatty acid is myristoleic acid.

44. The method according to claim 43, wherein the concentration of the myristoleic acid is greater than about 81.9 micrograms/ml.

45. The method according to claim 39, wherein the free fatty acids include 12-methyldodecanoic acid.

46. The method according to claim 45, wherein the concentration of the 12-methyldodecanoic acid is greater than about 49 micrograms/ml.

47. The method according to claim 39, wherein the free fatty acids include gamma-linolenic acid.

48. The method according to claim 47, wherein the concentration of the gamma-linolenic acid is greater than about 2.34 micrograms/ml.

49. A pharmaceutical composition comprising a free fatty acid in a pharmaceutically acceptable carrier.

50. The pharmaceutical composition according to claim 49, wherein the free acid is an unsaturated free fatty acid.

51. The pharmaceutical composition according to claim 49, wherein the free fatty acid is a saturated free fatty acid.

52. The pharmaceutical composition according to claim 49, wherein the free fatty acid is capric acid.

53. The pharmaceutical composition according to claim 49, wherein the free fatty acid is lauroleic acid.

54. The pharmaceutical composition according to claim 49, wherein the free fatty acid is myristoleic acid.

55. The pharmaceutical composition according to claim 49, wherein the free fatty acid is 12-methyldodecanoic acid.

56. The pharmaceutical composition according to claim 49, wherein the free fatty acid is gamma-linolenic acid.

57. The pharmaceutical composition according to claim 49, wherein the free fatty acid is lauric acid.

58. The pharmaceutical composition according to claim 49, wherein the free fatty acid is palmitoleic acid.

59. The pharmaceutical composition according to claim 49, wherein the free fatty acid is linoleic acid.

60. The pharmaceutical composition according to claim 49, wherein the free fatty acid is arachidonic acid.

61. The pharmaceutical composition according to claim 49, wherein the composition is for topical and/or systemic administration.

62. The method according to claim 1 aherein the concentration of the antimicrobial agent is in the micromolar range.

63. The method according to claim 1 wherein the concentration of the antimicrobial agent is about 100 micrograms/mi or lower.

64. The method according to claim 1 wherein the concentration of the antimicrobial agent is between about 1 microgram/mi and about 100 micrograms/mi.

65. The method according to claim 12 wherein the concentration of the unsaturated free fatty acids enriched fraction is between about 3.7 micro grams/mi and about 100 micrograms/mI.

66. The method according to claim 16 wherein the concentration of the capric acid is between about 1.55 micrograms/mi and about 100 micrograms/mi.

67. The method according to claim 18 wherein the concentration of the lauric acid is between about 1.8 micrograms/mi and about 100 micrograms/mi.

68. The method according to claim 20 wherein the concentration of the myristoleic acid is between about 2.04 micrograms/mi and about 100 micrograms/mi.

69. The method according to claim 22 wherein the concentration of the palmitoleic acid is between about 2.29 micrograms/mi and about 100 micrograms/mi.

70. The method according to claim 24 wherein the concentration of the linoleic acid is between about 22.7 micrograms/mi and about 100 micrograms/mi.

71. The method according to claim 26 wherein the concentration of the conjugated linoleic acid is between about 2.52 micrograms/mi and about 100 micrograms/mi.

72. The method according to claim 28 wherein the concentration of the arachidonic acid is between about 8.22 micrograms/mi and about 100 micrograms/mi.

73. The method according to claim 30 wherein the concentration of the gamma- linolenic acid is between about 25 micromolar and about 100 micromolar.

74. The method according to claim 39 wherein the concentration of the unsaturated free fatty acids enriched fraction is about 1 micrograms/mi to about 500 micrograms/mi.

75. The method according to claim 40 wherein the pathogen is Aspergillus fumigatus and the concentration of the capric acid is greater than about 127 micromolar and less than 1000 micromolar.

76. The method according to claim 40 wherein the concentration of the capric acid is greater than about 182 micromolar and less than about 1000 micromolar.

77. The method according to claim 40 wherein the pathogen is Candida albicans and the concentration of the capric acid is about 182 micromolar.

78. The method according to claim 43 wherein the pathogen is Candida albicans and the concentration of the myristoleic acid is greater than about 81.9 micromolar and less than about 1000 micromolar.

79. The method according to claim 43 wherein the pathogen is Aspergillus fumigatus and the concentration of the myristoleic acid is about 192 micromolar.

80. The method according to claim 43 wherein the pathogen is Aspergillus fumigatus and the concentration of the myristoleic acid is greater than about 192 micromolar but less than 1000 micromolar.

81. The method according to claim 45 wherein the pathogen is Candida albicans and the concentration of the 1 2-methyldodecanoic acid is greater than about 49 micromolar and less than about 1000 micromolar.

82. The method according to claim 45 wherein the pathogen is Aspergillus fumigatus and the concentration of the 12-methyldodecanoic acid is about 283 micromolar.

83. The method according to claim 45 wherein the pathogen is Aspergillus fumigatus and the concentration of the 12-methyldodecanoic acid is greater than about 283 micromolar and less than about 1000 micromolar.

84. The method according to claim 47 wherein the pathogen is Candida albicans and the concentration of the gamma-linolenic acid is greater than about 2.34 micromolar and less than about 1000 micromolar.

85. The method according to claim 47 wherein the pathogen is Aspergillus fumigatus and the concentration of the gamma-linolenic acid is about 13.4 micromolar.

86. The method according to claim 47 wherein the pathogen is Aspergillus fumigatus and the concentration of the gamma-linolenic acid is greater than about 13.4 micromolar and less than about 1000 micromolar.

87. The method according to claim 47 wherein the concentration of the gamma- linolenic acid is about 100 micromolar.

88. The method according to claim 47 wherein the concentration of the gamma- linolenic acid is greater than about 100 micromolar and less than about 1000 micromolar.
